SEQUENCE LISTING (10 sequences)

SEQ ID NO: 1 - DNA, 20 bp, Artificial Sequence
  Synthetic primer
tcaaggtttccaaggaccrg

SEQ ID NO: 2 - DNA, 20 bp, Artificial Sequence
  Synthetic primer
tcaaggtttccaaggacctg

SEQ ID NO: 3 - DNA, 20 bp, Artificial Sequence
  Synthetic primer
aaaggggagctggctacttc

SEQ ID NO: 4 - DNA, 20 bp, Artificial Sequence
  Synthetic primer
gcgagtaggagcagttggag

SEQ ID NO: 5 - DNA, 20 bp, Artificial Sequence
  Synthetic primer
tgaagaggggcacatgctga

SEQ ID NO: 6 - DNA, 20 bp, Artificial Sequence
  Synthetic primer
gtgggagttgggctgactcg

SEQ ID NO: 7 - DNA, 22 bp, Artificial Sequence
  Synthetic primer
gatcctggacctacacgacttc

SEQ ID NO: 8 - DNA, 22 bp, Artificial Sequence
  Synthetic primer
ggccttcagtacctttgtgaac

SEQ ID NO: 9 - DNA, 20 bp, Artificial Sequence
  Synthetic primer
gcacgaccttgagggcagcc

SEQ ID NO: 10 - DNA, 22 bp, Artificial Sequence
  Synthetic primer
catcgtggctaaacaggtactg

(19) United States
(12) Patent Application Publication
(10) Pub. No.: US 2019/0111108 A1
(43) Pub. Date: Apr. 18, 2019

(54) Composition Including GDF11 and Use Thereof

(71) Applicant: Kangstem Biotech Co., Ltd., Seoul (KR)

(72) Inventors: Yoon Jin Kim, Seoul (KR); Seung Hee Lee, Seoul (KR); Kwang Won Seo, Gyeonggi-do (KR); Kyung Sun Kang, Seoul (KR)

(21) Appl. No.: 16/075,262

(22) PCT Filed: Dec. 2, 2016
(86) PCT No.: PCT/KR2016/014138

§ 371 Date: Aug. 3, 2018

(30) Foreign Application Priority Data
Feb. 4, 2016 (KR) 10-2016-0014051

(51) Int. Cl. A61K 38/18; A61K 8/64; C12N 5/077; A61Q 19/08; A61P 17/02; A61K 35/28; A61K 35/51; A61K 8/98

(52) U.S. Cl. CPC A61K 38/18; A61K 8/64; C12N 5/0656; A61Q 19/08; C12N 2501/19; A61K 35/28; A61K 35/51; A61K 8/981; A61P 17/02

(57) ABSTRACT

Provided are a pharmaceutical composition for regenerating skin, a pharmaceutical composition for improving wrinkles, a pharmaceutical composition for treating wounds, a quasi-drug composition for regenerating skin, a quasi-drug composition for improving wrinkles, a quasi-drug composition for treating wounds, a cosmetic composition for regenerating skin, a cosmetic composition for improving wrinkles, a cosmetic composition for improving wounds, and a medium composition for culturing fibroblasts, each composition including GDF11 or a human-derived adult stem cell culture medium including the same, a method of culturing fibroblasts by using the medium composition, and a method of preparing GDF11 by culturing stem cells. The GDF11 provided in the present invention may be included in a human-derived adult stem cell culture medium to exhibit an effect of promoting fibroblast proliferation, and may thereby be widely applied to the development of a variety of products for skin regeneration, wrinkle improvement, or wound treatment.

### TECHNICAL FIELD

The present invention relates to a composition including GDF11 and use thereof, and more particularly, the present invention relates to a pharmaceutical composition for regenerating skin, a pharmaceutical composition for improving wrinkles, a pharmaceutical composition for treating wounds, a quasi-drug composition for regenerating skin, a quasi-drug composition for improving wrinkles, a quasi-drug composition for treating wounds, a cosmetic composition for regenerating skin, a cosmetic composition for improving wrinkles, and a cosmetic composition for improving wounds, each composition including GDF11 or a human-derived adult stem cell culture medium including the same, a method of regenerating skin, a method of improving wrinkles, and a method of treating wounds, each method including the step of administering the composition, a medium composition for culturing fibroblasts, a method of culturing fibroblasts using the medium composition, and a method of preparing GDF11 by culturing the stem cells.

### BACKGROUND ART

Recently, modern people's interest in healthy living is increasing, and due to improvement of standards of living, women's advancement in society, and changes to an aging society, etc., consumers' desire for cosmetics is gradually changing from cosmetics simply for the enhancement of personal appearance to cosmetics which emphasize functional aspects. Therefore, studies are being actively conducted to find natural substances which are harmless to the human body. Skin aging is a complex biological phenomenon, and may be largely divided into two components; natural aging (endogenous aging) that occurs over time and photoaging caused by external factors, especially ultraviolet radiation. Skin is always exposed to oxygen and sunlight, and oxidative stress resulting therefrom promotes skin aging. Since skin is constantly in contact with various environmental factors, it is directly exposed to the attack of oxidative stressors. Excessive exposure of skin to UV rays generates a large amount of reactive oxygen species (ROS) in the skin, and the antioxidant defense system becomes unbalanced, eventually promoting aging.

Elastase, present in the neutrophil granules of the human body, is an enzyme that degrades elastin, which is an important matrix protein, to maintain skin elasticity in the dermis, and is a non-specific hydrolase capable of degrading collagen, which is another important matrix protein. Inhibitors of elastase exhibit an effect of improving skin wrinkles, and ursolic acid, etc. is used as an elastase inhibitor. However, since ursolic acid is insoluble in solvents such as water or oil, it is difficult to formulate, and generally, there is difficulty in using ursolic acid.

Collagen is mostly found in dermal layers of the skin and accounts for about 70% to 80% of the dry weight of the skin. Collagen, which is a major structural element of the extracellular matrix, is a major matrix protein generated in fibroblasts of skin. Synthesis and degradation of collagen are properly controlled, but it is synthesized less with age. UV radiation promotes expression of collagenase, which is a collagen-degrading enzyme and is known to be closely related with wrinkle formation in the skin.

Further, deficiency of matrix proteins is one of the major causes of photoaging. UV radiation decreases synthesis of collagen and elastin, which are components filling the space between cells, and increases expression of various proteolytic enzymes of matrix proteins. Therefore, matrix proteins are degraded by collagenase and elastase, which are collagen- and elastin-degrading enzymes, as the major causes of skin aging. The dermal layer plays an important role in determining physicochemical properties of skin and nourishing capillaries and epidermis, and thus is closely related to skin aging.

Generally, products obtained by blending collagen with a skin external composition such as cosmetics or ointments are brought into the market to take advantage of the wrinkle-improving effect of collagen. However, since the collagen in these products is a large molecule, it cannot be absorbed percutaneously simply by application to the skin, and thus a wrinkle-improving effect cannot be expected. In order to solve this problem, a material that can promote the synthesis of collagen has attracted much attention. Examples of generally known collagen synthesis-promoting materials include vitamin C, retinoic acid, a transforming growth factor (TGF), protein originating from animal placenta (JP8-231370), betulinic acid (JP8-208424), a chlorella extract (promoting proliferation of fibroblasts, JP9-40523 and JP10-36283), etc.

In recent years, interest in stem cells has increased, and it was reported that stem cells having differentiation potential or extracts thereof may inhibit skin aging. Accordingly, research has been actively conducted on methods of inhibiting skin aging using stem cells. For example, Korean Patent Publication No. 2009-0116659 discloses a cosmetic composition including a stem cell culture medium for wrinkle improvement, whitening, or anti-aging. However, since the stem cell culture medium contains numerous components, it is not yet known what components substantially exhibit the wrinkle-improving effect. If active components capable of exhibiting the wrinkle-improving effect are identified, products capable of more effectively improving wrinkles are expected to be developed. However, there has been no satisfactory outcome.

### DISCLOSURE

### Technical Problem

The present inventors have made many efforts to identify active ingredients capable of improving skin wrinkles from a human-derived adult stem cell culture medium which is known to have the wrinkle-improving effect, and as a result, they found that GDF11 included in the culture medium promotes fibroblast proliferation, increases a synthesis level of collagen which improves skin wrinkles, and suppresses activity of collagenase, and thus GDF11 is an active ingredient to exhibit the wrinkle-improving effect, thereby completing the present invention.

### Technical Solution

An object of the present invention is to provide a pharmaceutical composition for regenerating skin including GDF11 or a human-derived adult stem cell culture medium including the same.

Another object of the present invention is to provide a pharmaceutical composition for improving wrinkles including GDF11 or the human-derived adult stem cell culture medium including the same.

Still another object of the present invention is to provide a pharmaceutical composition for treating wounds including GDF11 or the human-derived adult stem cell culture medium including the same.

Still another of the present invention is to provide a quasi-drug composition for regenerating skin including GDF11 or the human-derived adult stem cell culture medium including the same.

Still another of the present invention is to provide a quasi-drug composition for improving wrinkles including GDF11 or the human-derived adult stem cell culture medium including the same.

Still another object of the present invention is to provide a quasi-drug composition for treating wounds including GDF11 or the human-derived adult stem cell culture medium including the same.

Still another of the present invention is to provide a cosmetic composition for regenerating skin including GDF11 or the human-derived adult stem cell culture medium including the same.

Still another of the present invention is to provide a cosmetic composition for improving wrinkles including GDF11 or the human-derived adult stem cell culture medium including the same.

Still another object of the present invention is to provide a cosmetic composition for improving wounds including GDF11 or the human-derived adult stem cell culture medium including the same.

Still another object of the present invention is to provide a medium composition for culturing fibroblasts including GDF11 or the human-derived adult stem cell culture medium including the same.

Still another object of the present invention is to provide a method of culturing fibroblasts by using the medium composition.

Still another object of the present invention is to provide a method of preparing GDF11 including the step of culturing the human-derived adult stem cells.

Still another object of the present invention is to provide a method of treating wounds including the step of administering to a subject the composition including GDF11 or the human-derived adult stem cell culture medium including the same.

Still another object of the present invention is to provide a method of regenerating skin including the step of administering to a subject the composition including GDF11 or the human-derived adult stem cell culture medium including the same.

Still another object of the present invention is to provide a method of improving wrinkles including the step of administering to a subject the composition including GDF11 or the human-derived adult stem cell culture medium including the same.

Still another object of the present invention is to provide use of GDF11 or the human-derived adult stem cell culture medium including the same in the wound treatment, skin regeneration, or wrinkle improvement.

### Advantageous Effects

The GDF11 provided in the present invention may be included in a human-derived adult stem cell culture medium to exhibit an effect of promoting fibroblast proliferation, and may thereby be widely applied to the development of a variety of products for skin regeneration, wrinkle improvement, or wound treatment.

### DESCRIPTION OF DRAWINGS

FIG. 1 shows photographs (A) showing results of comparing effects of a control group (CTL), a fibroblast culture medium (HDF CM), a human adipose-derived stem cell culture medium (AD-MSC CM), or a human umbilical cord blood-derived mesenchymal stem cell culture medium (UCB-MSC CM) on proliferation ability of fibroblasts, a graph (B) showing absorbance, a graph (C) showing the number of cells, and a graph (D) showing a total protein expression level;

FIG. 2 shows microscopic images showing results of comparing effects of the control group (CTL), the fibroblast culture medium (HDF CM), the human adipose-derived stem cell culture medium (AD-MSC CM), or the human umbilical cord blood-derived mesenchymal stem cell culture medium (UCB-MSC CM) on migration of fibroblasts;

FIG. 3A shows a Western blot analysis image showing results of comparing effects of the control group (CTL), the fibroblast culture medium (HDF CM), the human adipose-derived stem cell culture medium (AD-MSC CM), or the human umbilical cord blood-derived mesenchymal stem cell culture medium (UCB-MSC CM) on protein expression levels of various matrix proteins expressed in the fibroblasts;

FIG. 3B shows an RT-PCR analysis image showing the results of comparing the effects of the control group (CTL), the fibroblast culture medium (HDF CM), the human adipose-derived stem cell culture medium (AD-MSC CM), or the human umbilical cord blood-derived mesenchymal stem cell culture medium (UCB-MSC CM) on expression levels of various matrix proteins expressed in the fibroblasts;

FIG. 4A shows a graph and an image showing results of comparing therapeutic effects on wounds of the control group (CTL), the fibroblast culture medium (HDF CM), the human adipose-derived stem cell culture medium (AD-MSC CM), or the human umbilical cord blood-derived mesenchymal stem cell culture medium (UCB-MSC CM);

FIG. 4B shows tissue images showing results of comparing the wound areas of wound animal models, each animal treated with the control group (CTL), the fibroblast culture medium (HDF CM), the human adipose-derived stem cell culture medium (AD-MSC CM), or the human umbilical cord blood-derived mesenchymal stem cell culture medium (UCB-MSC CM);

FIG. 5A shows a graph showing RT-PCR and real-time qPCR results of comparing GDF11 mRNA expression levels in fibroblasts (HDF), human adipose-derived stem cells (AD-MSC), or human umbilical cord blood-derived mesenchymal stem cells (UCB-MSC), and FIG. 5B shows a photograph showing the result of RT-PCR;

FIG. 6A shows Western blot analysis images showing results of comparing levels of GDF11 in the human bone marrow-derived stem cell culture medium (BM-MSC CM) used as a control group (CTL), the human adipose-derived stem cell culture medium (AD-MSC CM), or the human umbilical cord blood-derived stem cell culture medium (UCB-MSC CM), and FIG. 6B shows a quantification graph of the Western blot analysis results;

FIG. 7A shows a graph showing the effect of GDF11 on proliferation of human umbilical cord blood-derived mesenchymal stem cells, and FIG. 7B shows a photograph showing results of comparing changes in the collagen expression level according to suppression of GDF11 expression in the human umbilical cord blood-derived mesenchymal stem cells;

FIG. 8 shows graphs showing results of comparing changes in the GDF11 expression level in the human umbilical cord blood-derived mesenchymal stem cells treated with the control group (a), EGF (b), bFGF (c), TGF-betal (d), or vEGF (e) according to treatment time and concentration; and

FIG. 9 shows graphs and a photograph showing results of comparing fibroblast proliferation levels in the GDF11-treated fibroblasts (FIG. 9A), expression levels of type I collagen expressed in the fibroblasts (FIGS. 9B and 9F), expression levels of type III collagen expressed in the fibroblasts (FIGS. 9C and 9F), expression levels of elastin expressed in the fibroblasts (FIGS. 9D and 9F), and expression levels of MMP1 expressed in the fibroblasts (FIGS. 9E and 9F).

### BEST MODE

The present inventors conducted various studies to identify active ingredients capable of improving skin wrinkles from a human-derived adult stem cell culture medium which is known to have a wrinkle-improving effect, and they focused on GDF11 (growth differentiation factor 11), which is known to have an effect in preventing or treating dementia. GDF11 is known as a protein that restores motor ability and regenerates degenerative cerebral blood vessels. The present inventors found that GDF11 is detected in a human-derived adult stem cell culture medium that promotes proliferation and migration of fibroblasts and increases expression of matrix proteins, and as a result of comparison of the human-derived adult stem cell culture with other stem cell culture media, it was confirmed that a large amount of GDF11 was included in a culture medium obtained by culturing human umbilical cord blood-derived mesenchymal stem cells among various adult stem cells. Accordingly, effects of GDF11 were investigated, and as a result, it was confirmed that when GDF11 expression is decreased, a proliferation rate of human-derived adult stem cells decreases, and an expression level of type III collagen expressed therefrom is decreased. It was also confirmed that a variety of growth factors (EGF, bFGF, TGF-betal, or vEGF) are involved in GDF11 expression, and treatment of fibroblasts with GDF11 promotes proliferation of fibroblasts, and in these fibroblasts, collagen and elastin expression is increased. Accordingly, it was demonstrated that GDF11 may promote wound-healing, skin-regenerating, and wrinkle-improving effects which may be induced by promoting fibroblast proliferation.

Until now, it had been never disclosed that GDF11 is involved in fibroblast proliferation and ultimately in the treatment of skin wounds, improvement of wrinkles, and regeneration of skin, which was first demonstrated by the present inventors.

In order to achieve the above-described object, an aspect of the present invention provides a pharmaceutical composition for regenerating skin, improving wrinkles, or treating wounds, including GDF11 or a human-derived adult stem cell culture medium including the same.

As used herein, the term “GDF11 (growth differentiation factor 11)” is also called “BMP-11 (bone morphogenetic protein 11)” and refers to a protein expressed by GDF11 gene located on human chromosome 12. GDF11 is known as a myostatin analogue protein and is known to act as an inhibitor that inhibits neuronal growth. Recently, it was reported that GDF11 restores motor ability and regenerates degenerative cerebral blood vessels to exhibit an effect of preventing or treating dementia. Sequence information of GDF11 of the present invention is available from a known database such as the National Center for Biotechnology Information (NCBI), etc., for example, human-derived GDF11 gene (NM_005811), human-derived GDF11 protein (NP_005802), mouse-derived GDF11 gene (NM_010272), mouse-derived GDF11 protein (NP_034402), etc.

In the present invention, GDF11 may exhibit skin-regenerating, wrinkle-improving, or wound-healing effects through enhancement of fibroblast proliferation, and therefore, GDF11 may be used as an active ingredient of a composition exhibiting the above effects.

As used herein, the term “human-derived adult stem cell culture medium” refers to a culture obtained by culturing human adult stem cells or a culture supernatant obtained by removing stem cells from the culture. The culture medium obtained by culturing adult stem cells includes various substances (e.g., GDF1, etc.) which are secreted during culturing of the adult stem cells, and therefore, when the human-derived adult stem cell culture medium is used, the effects of regenerating skin, improving wrinkles, and treating wounds may be obtained through enhancement of fibroblast proliferation.

In the present invention, the human-derived adult stem cell culture medium may be interpreted as a culture supernatant obtained by culturing human adult stem cells, and the adult stem cells used herein are not particularly limited, as long as they are able to secrete GDF11 into the culture medium. For example, the adult stem cells may be those derived from umbilical cord, umbilical cord blood, bone marrow, fat, muscle, nerve, skin, amniotic membrane, or placenta. In the present invention, a culture medium of human umbilical cord blood-derived mesenchymal stem cells was used as the human-derived mesenchymal stem cell culture medium.

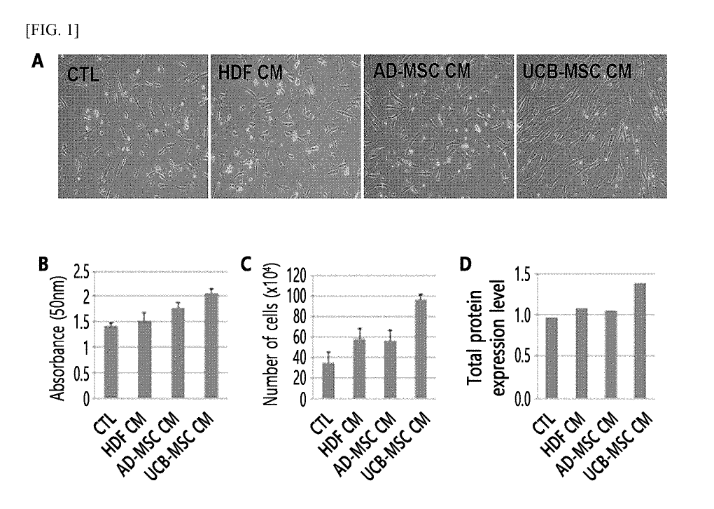

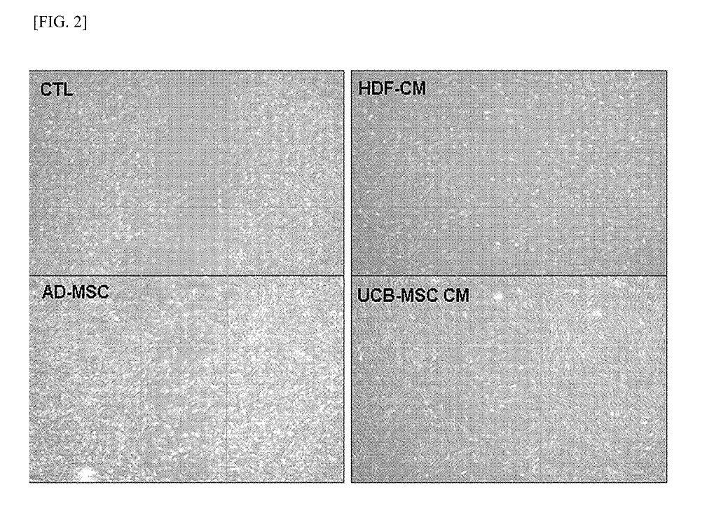

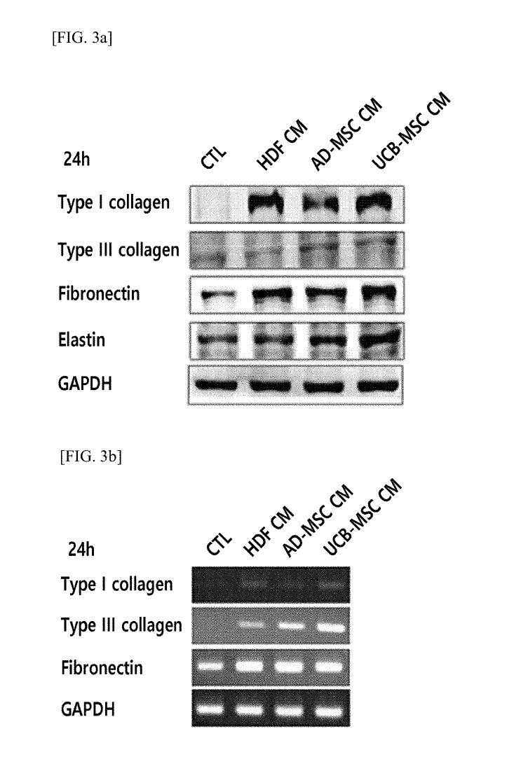

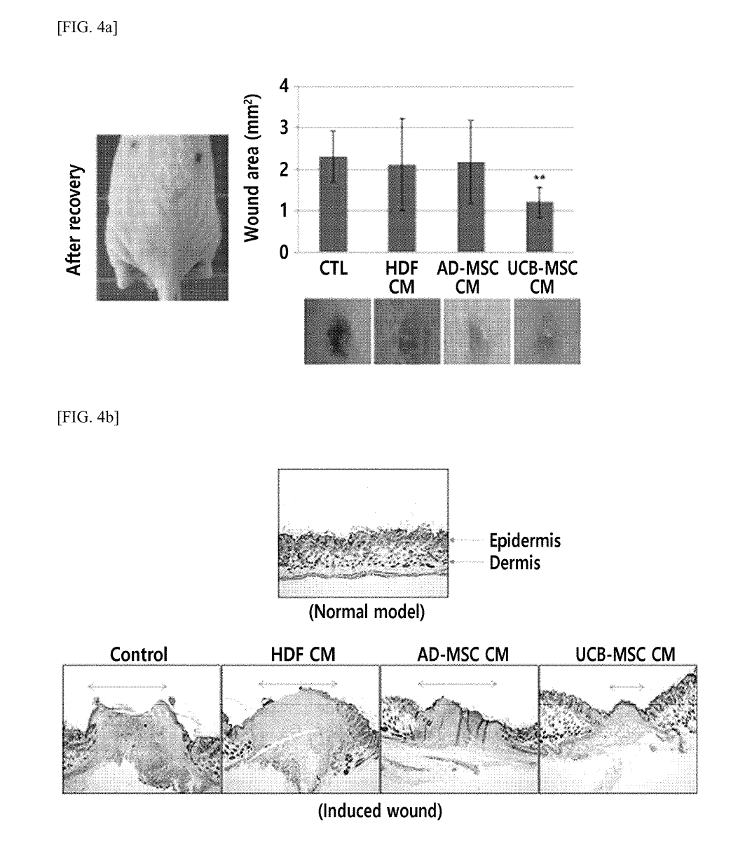

According to an embodiment of the present invention, an effect of the stem cell culture medium on proliferation ability of fibroblasts was examined, and as a result, it was confirmed that the human umbilical cord blood-derived mesenchymal stem cell culture medium (UCB-MSC CM) may promote fibroblast proliferation and may also increase a total amount of proteins secreted from the cells (FIG. 1), and may promote migration of fibroblasts (FIG. 2), as compared with a fibroblast culture medium (HDF CM) or a human adipose-derived stem cell culture medium (AD-MSC CM). It was also confirmed that the human umbilical cord blood-derived mesenchymal stem cell culture medium (UCB-MSC CM) may increase expression levels of various matrix proteins (collagen, fibronectin, elastin, etc.) expressed in fibroblasts to relatively high levels (FIGS. 3A and 3B), and may exhibit a relatively excellent wound-healing effect in an animal model (FIGS. 4A and 4B).

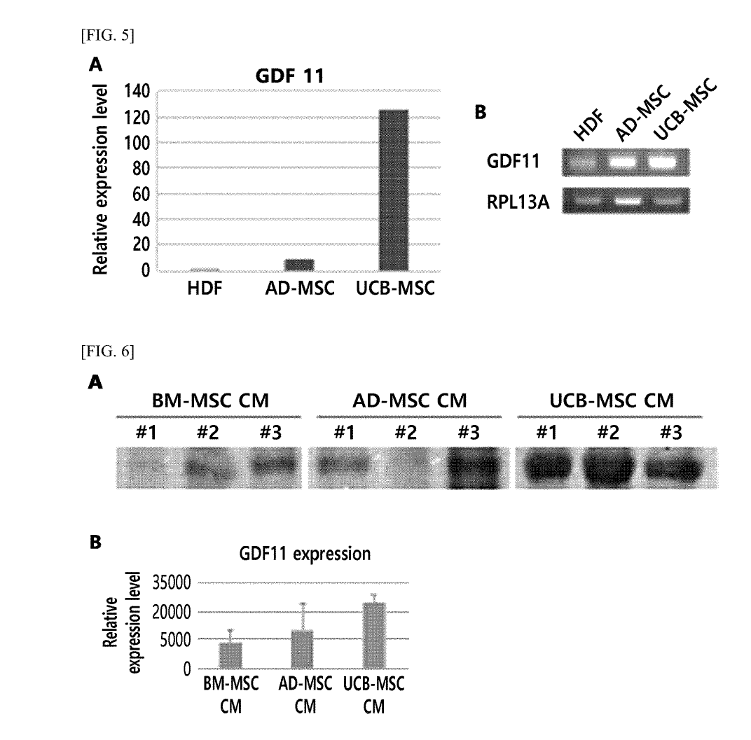

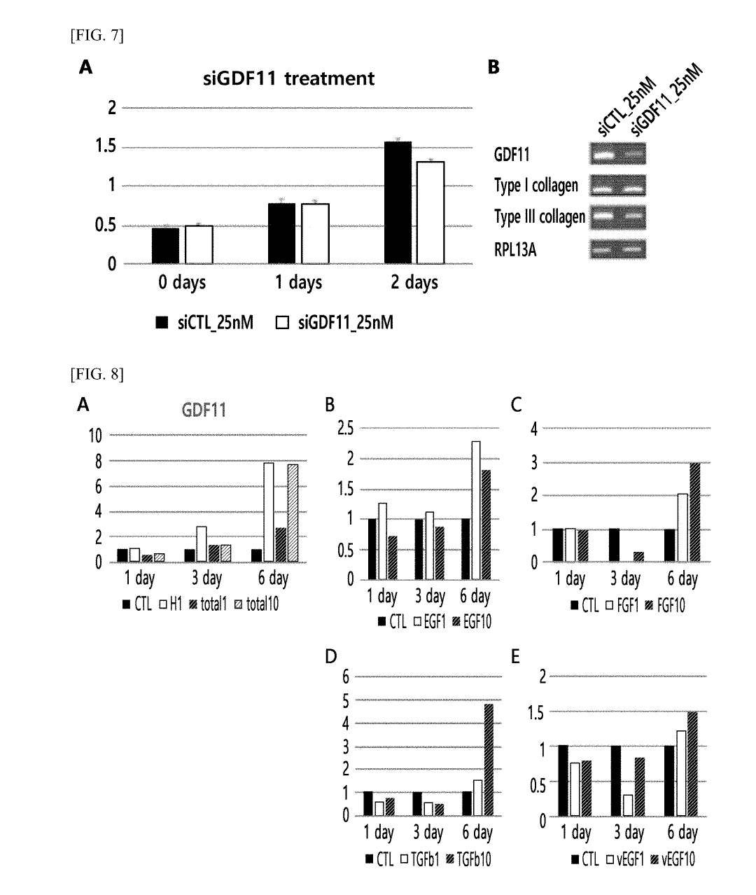

GDF11 (growth differentiation factor 11), which is an active ingredient of the human-derived adult stem cell culture medium showing the above effects, was analyzed for the effects, and as a result, it was confirmed that a large amount of GDF11 is included in the human umbilical cord blood-derived mesenchymal stem cell culture medium (FIGS. 5A and 5B), and GDF11 is secreted at a higher level in the human umbilical cord blood-derived mesenchymal stem cells than human bone marrow- or adipose-derived mesenchymal stem cells (FIGS. 6A and 6B). Further, the effect of GDF11 on the human umbilical cord blood-derived mesenchymal stem cells was analyzed, and as a result, it was confirmed that when the expression level of GDF11 is suppressed, proliferation of the human umbilical cord blood-derived mesenchymal stem cells is suppressed, and the expression level of type III collagen expressed therefrom is decreased (FIGS. 7A and 7B), and EGF, bFGF, TGF-betal, and vEGF are involved in the expression of GDF11 (FIG. 8).

The effect of GDF11 on fibroblasts was analyzed, and as a result, it was confirmed that GDF11 promotes fibroblast proliferation and increases collagen and elastin expression in the fibroblasts.

Accordingly, it was analyzed that GDF11 may promote the wound-healing, skin-regenerating, wrinkle-improving effects which may be induced by enhancement of fibroblast proliferation.

The pharmaceutical composition of the present invention may be prepared in a form of a pharmaceutical composition for regenerating skin, improving wrinkles, and treating wounds by further including an appropriate carrier, excipient, or diluent commonly used in the preparation of pharmaceutical compositions. Specifically, the pharmaceutical composition may be formulated according to a common method in oral dosage forms, including powders, granules, tablets, capsules, suspensions, emulsions, syrup, aerosol, etc., preparations for external application, suppositories, and sterile injectable solutions. In the present invention, the carrier, excipient, and diluent that may be included in the pharmaceutical composition may be exemplified by lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxylbenzoate, talc, magnesium stearate, mineral oil, etc. The pharmaceutical composition of the present invention may be formulated with commonly used diluents or excipients, such as fillers, extenders, binders, wetting agents, disintegrants, surfactants, etc. Solid formulations for oral administration may include tablets, pills, powders, granules, capsules, etc., and such solid formulations may be prepared by mixing the GDF11 or human-derived adult stem cell culture medium including the same with at least one excipient, for example, starch, calcium carbonate, sucrose, lactose, gelatin, etc. In addition to simple excipients, lubricants such as magnesium stearate or talc may also be used. Liquid formulations for oral administration may include suspensions, solutions for internal use, emulsions, syrup, etc., and may include various excipients, for example, wetting agents, flavoring agents, aromatics, preservatives, etc., in addition to water and liquid paraffin, which are frequently used simple diluents. Formulations for parenteral administration may include sterilized aqueous solutions, non-aqueous solutions, suspensions, emulsions, lyophilized preparations, suppositories, etc. As non-aqueous solvents or suspending agents, propylene glycol, polyethylene glycol, plant oils such as olive oil, injectable esters such as ethyl oleate, etc. may be used. As a base of the suppositories, witepsol, Macrogol, Tween 61, cacao butter, laurin butter, glycerogelatin, etc. may be used.

A content of GDF11 in the pharmaceutical composition of the present invention may be, but is not particularly limited to, 1×10⁻⁹% by weight to 50% by weight, more preferably 0.01% by weight to 20% by weight, based on the total weight of the final composition.

The pharmaceutical composition of the present invention may be administered in a pharmaceutically effective amount, and the term “pharmaceutically effective amount”, as used herein, means an amount which is sufficient to treat or prevent diseases at a reasonable benefit/risk ratio applicable to any medical treatment or prevention. The effective dosage level may be readily determined depending on factors including severity of a disease, activity of a drug, a patient's age, body weight, health conditions, sex, and drug sensitivity, administration time, administration route, and excretion rate of the composition of the present invention, duration of treatment, drugs used in combination or used concurrently with the composition of the present invention, and other factors known in the medical field. The pharmaceutical composition of the present invention may be administered alone or in combination with any known pharmaceutical composition for regenerating skin, improving wrinkles, or treating wounds. It is important to administer an amount to obtain the maximum effect with a minimum amount without adverse effects, considering all of the factors described above.

The administration dose of the pharmaceutical composition of the present invention may be determined by those skilled in the art considering the purpose of use, severity of the disease, a patient's age, body weight, sex, anamnesis, or a kind of material(s) to be used as an active ingredient, etc. For example, the pharmaceutical composition of the present invention may be administered in an amount of about 0.1 ng/kg to about 100 mg/kg, preferably about 1 ng/kg to about 10 mg/kg per adult, and the administration frequency of the pharmaceutical composition of the present invention may be administered once a day or several times in divided doses a day, but is not particularly limited thereto. The above administration dose does not limit the scope of the present invention in any aspect.

Another aspect of the present invention provides a method of treating wounds, the method including the step of administering to a subject the pharmaceutical composition in a pharmaceutically effective amount.

The “subject”, as used herein, may include without limitation all mammalian animals including mice, livestock, etc., or cultured fish, etc. in need of skin regeneration, wrinkle improvement, or wound treatment.

The “treatment”, as used herein, refers to all kinds of actions associated with skin regeneration, wrinkle improvement, or wound treatment, or advantageous changes due to administration of the pharmaceutical composition including GDF11 of the present invention as an active ingredient to a subject in need of skin regeneration, wrinkle improvement, or wound treatment.

The pharmaceutical composition for regenerating skin, improving wrinkles, or treating wounds of the present invention may be administered via any of the common routes, as long as it is able to reach a desired tissue. The pharmaceutical composition of the present invention may be, but is not particularly limited to, administration intraperitoneally, intravenously, intramuscularly, subcutaneously, intradermally, orally, intranasally, intrapulmonarily, or intrarectally according to the desired purpose. However, since GDF11 may be denatured or degraded by gastric acid upon oral administration, active ingredients of a composition for oral administration should be coated or formulated for protection against degradation in the stomach. In addition, the composition may be administered using a certain apparatus capable of transporting the active ingredient into a target cell.

Still another aspect of the present invention provides a quasi-drug composition for regenerating skin, improving wrinkles, or treating wounds including GDF11 or the human-derived adult stem cell culture medium including the same.

The term “improvement”, as used herein, refers to all kinds of actions that at least reduce parameters related to a condition to be treated, for example, a degree of a symptom.

The term “quasi-drug”, as used herein, refers to an article having a milder action than drugs, among articles being used for the purpose of diagnosis, treatment, improvement, alleviation, handling, or prevention of human or animal diseases. For example, according to the Pharmaceutical Affairs Law, the quasi-drugs are those, excluding articles used as drugs, including articles made from fiber or rubber which are used for the purpose of treating or preventing human or animal diseases, other than a tool or a machine, or an analogue thereof, which have a mild action on or have no direct influence on the human body, and articles which are used for the purpose of disinfection or pest control for the prevention of infectious diseases. The kind or formulation of the quasi-drug composition of the present invention is, but is not particularly limited to, preferably, disinfectants, shower foams, mouthwash, wet tissues, detergent soap, hand wash, humidifier fillers, masks, ointments, a filter coating, etc.

Still another aspect of the present invention provides a cosmetic composition for regenerating skin, improving wrinkles, or improving wounds, including GDF11 or the human-derived adult stem cell culture medium including the same.

As described above, since GDF11 may promote fibroblast proliferation, it may be used in the preparation of cosmedical products exhibiting the skin-regenerating, wrinkle-improving, or wound-improving effects which may be induced by fibroblast proliferation.

The term “cosmedical (cosmeceutical) product”, as used herein, refers to a functional product which is prepared by introducing a cosmetic with a specialized therapeutic function of a medicine so as to have a specialized function by enhancing a physiological efficacy or effect, unlike general cosmetics. The cosmedical product refers to a product that assists in skin whitening, a product that assists in skin wrinkle improvement, and a product that assists in tanning the skin or protecting the skin from UV rays, each product determined by the Ordinance of the Ministry of Health and Welfare.

In the present invention, the cosmedical product refers to a product that helps prevent skin aging by exhibiting the skin-regenerating, wrinkle-improving, and wound-improving effects, among various cosmedical products. For example, the cosmedical product may be a cosmedical product including GDF11 or the human-derived adult stem cell culture medium including the same as an active ingredient, but is not particularly limited thereto. A content of GDF11 in the cosmedical product is not particularly limited.

The cosmedical product of the present invention may include GDF11 or the human-derived adult stem cell culture medium including the same as an active ingredient, and may further include commonly used cosmetic materials. For example, for an aqueous skin formulation, glycerol, propylene glycol, 1,3-butylene glycol, sorbitol, polyethylene glycol, carboxyvinyl polymers, xanthan gum, carboxymethyl cellulose, hydroxyethylcellulose, hydroxymethylcellulose, locust bean gum, allantoin, carrageenan, etc. may be added; wax, paraffin wax, stearyl alcohol, carnauba wax, candelilla wax and calcium stearate, aluminum stearate, zinc stearate, witchhazel, etc. may be used as a viscosity and hardness regulator; butyl methoxydibenzoyl methane, octyl methoxycinnamate, etc. may be used as a UV absorber; an extender pigment such as titanium dioxide, particulate titanium dioxide, kaolin, nylon powder, talc, sericite, mica, polymethylmethacrylate, etc. and a coloring pigment such as yellow iron oxide, black iron oxide, red iron oxide, ultramarine, chromium oxide, chromium hydroxide, etc. may be used as a pigment; 1,3-butylene glycol, concentrated glycerin, ethylene glycol, and a natural moisturizing agent such as chitin, chitosan, hyaluronic acid, lactic acid, glycolic acid, etc. may be used as a moisturizing agent; para-hydroxybenzoic acid esters, imidazolidinyl urea, etc. may be used as a preservative. These components may be used alone or in a combination of two or more thereof according to characteristics of products.

The cosmedical product of the present invention may be formulated in any form commonly prepared in the art, which is exemplified by a solution, a suspension, an emulsion, a paste, a gel, a cream, a lotion, a powder, a soap, a surfactant-containing cleansing, an oil, a powdered foundation, an emulsified foundation, a wax foundation, and a spray, but is not limited thereto. More specifically, the cosmedical product of the present invention may be prepared in the form of a soft lotion, a nutrition lotion, a nutrition cream, a massage cream, an essence, an eye cream, a cleansing cream, a cleansing foam, a cleansing water, a pack, a spray, or a powder.

When the formulation of the present invention is a paste, a cream, or a gel, animal oil, vegetable oil, wax, paraffin, starch, tragacanth, a cellulose derivative, polyethylene glycol, silicone, bentonites, silica, talc, zinc oxide, etc. may be used as a carrier component.

When the formulation of the present invention is a powder or spray, lactose, talc, silica, aluminum hydroxide, calcium silicate, or polyamide powders may be used as a carrier component. In particular, when the formulation of the present invention is a spray, a propellant such as chlorofluorohydrocarbon, propane/butane, or dimethyl ether may be additionally included.

When the formulation of the present invention is a solution or an emulsion, a solvent, a solubilizer, or an emulsifying agent may be used as a carrier component. For example, water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan may be used.

When the formulation of the present invention is a suspension, a liquid diluent such as water, ethanol, or propylene glycol; a suspension such as ethoxylated isostearyl alcohol, polyoxyethylene sorbitol ester, and polyoxyethylene sorbitan ester; microcrystalline cellulose, aluminum meta-hydroxide, bentonite, agar, or tragacanth may be used as a carrier component.

When the formulation of the present invention is a surfactant-containing cleansing, aliphatic alcohol sulfate, aliphatic alcohol ether sulfate, sulfosuccinate monoester, isethionate, an imidazolium derivative, methyl taurate, sarcosinate, fatty acid amide ether sulfate, alkylamidobetaine, aliphatic alcohol, fatty acid glyceride, fatty acid diethanolamide, vegetable oil, a lanolin derivative, ethoxylated glycerol fatty acid ester, etc. may be used as a carrier component.

Still another aspect of the present invention provides a medium composition for culturing fibroblasts including GDF11 or the human-derived adult stem cell culture medium including the same, and a method of culturing fibroblasts by using the medium composition.

As described above, since GDF11 or the human-derived adult stem cell culture medium including the same provided in the present invention may promote fibroblast proliferation, GDF11 or the human-derived adult stem cell culture medium including the same may be used as an active ingredient of the medium composition for culturing fibroblasts, and fibroblasts may be cultured by using the medium composition for culturing fibroblasts.

Meanwhile, the method of culturing fibroblasts provided in the present invention may include the step of seeding and culturing fibroblasts in the medium composition for culturing fibroblasts.

Still another aspect of the present invention provides a method of preparing GDF11 including the step of culturing the human-derived adult stem cells.

Specifically, the method of preparing GDF11 provided in the present invention may include the steps of (a) culturing the human-derived adult stem cells to obtain a culture supernatant; and (b) collecting GDF11 from the obtained culture supernatant.

The term “culturing”, as used herein, refers to an overall action that allows cells to grow under artificially controlled environmental conditions.

With respect to the objects of the present invention, the culturing may be performed in order to prepare GDF11 which is secreted from stem cells provided in the present invention into the culture supernatant, and the culturing method is not particularly limited, and a method widely known in the art may be used.

The culturing conditions are, but are not particularly limited to, pH (pH 5 to pH 9, preferably pH 6 to pH 8, most preferably pH 6.8) which may be adjusted with a basic compound (e.g., sodium hydroxide, potassium hydroxide, or ammonia), or an acidic compound (e.g., phosphoric acid or sulfuric acid), and an aerobic condition which may be maintained by injecting oxygen or an oxygen-containing gas into the culture medium. A culturing temperature is, but is not particularly limited to, for example, 30° C. to 40° C., for another example, 33° C. to 37° C., and for still another example, 37° C. A culturing time is also, but is not particularly limited to, for example, 5 days to 15 days, for another example, 7 days to 10 days, and for still another example, 7 days.

Furthermore, a medium used for the culturing may use sugars and carbohydrates (e.g., glucose, sucrose, lactose, fructose, maltose, molasses, starch, and cellulose) as a carbon source, oil and fat (e.g., soybean oil, sunflower oil, peanut oil, and coconut oil), a fatty acid (e.g., palmitic acid, stearic acid, and linolenic acid), an alcohol (e.g., glycerol and ethanol), and an organic acid (e.g., acetic acid) alone or in a mixture thereof; a nitrogen-containing organic compound (e.g., peptone, yeast extract, meat extract, malt extract, corn steep liquor, soy meal, and urea) or an inorganic compound (e.g., ammonium sulfate, ammonium chloride, ammonium phosphate, ammonium carbonate, and ammonium nitrate) as a nitrogen source alone or in a mixture thereof; potassium dihydrogen phosphate, dipotassium hydrogen phosphate, or corresponding sodium-containing salts thereof as a phosphorous source alone or in a mixture thereof; and other substances essential for growth such as metal salts (e.g., magnesium sulfate or iron sulfate), amino acids, and vitamins.

The medium may further include various growth factors such as EGF, bFGF, vEGF, TGF-β1, etc. in order to promote proliferation of stem cells.

Furthermore, the step of collecting GDF11 from the culture may be performed by a method known in the art. Specifically, the known method of collecting GDF11 used may preferably be, but is not particularly limited to, centrifugation, filtration, extraction, spraying, drying, evaporation, precipitation, crystallization, electrophoresis, fractional dissolution (e.g., ammonium sulfate precipitation), or chromatography (e.g., ion exchange, affinity, hydrophobic, and size exclusion).

Still another aspect provides a method of regenerating skin including the step of administering to a subject the composition including GDF11 or the human-derived adult stem cell culture medium including the same.

Still another aspect provides a method of improving wrinkles including the step of administering to a subject the composition including GDF11 or the human-derived adult stem cell culture medium including the same.

Still another aspect provides use of GDF11 or the human-derived adult stem cell culture medium including the same in the wound treatment, skin regeneration, or wrinkle improvement.

### Mode for Invention

Hereinafter, the present invention will be described in more detail with reference to Examples. However, these Examples are for illustrative purposes only, and the scope of the present invention is not intended to be limited by these Examples.

### EXAMPLE 1

### Acquisition of Stem Cell Culture Medium

### EXAMPLE 1-1

### Acquisition of Human Umbilical Cord Blood-Derived Mesenchymal Stem Cell Culture Medium

Human umbilical cord blood stem cells (1.89×10⁵ cells) isolated from umbilical cord blood were inoculated in EGM-2 (endothelial growth medium) containing 10% FBS, and cultured under conditions of 37° C. and 5% CO₂ for 48 hours to obtain cultured cells. The obtained cells were inoculated in H1 medium and cultured for 96 hours to obtain a human umbilical cord blood-derived mesenchymal stem cell culture medium. In this regard, a DMEM medium containing EGF, bFGF, vEGF, and TGF-β1 was used as the H1 medium.

### EXAMPLE 1-2

### Acquisition of Human Adipose-Derived Stem Cell Culture Medium

An aspirated adipose tissue was washed with PBS, and 1 μL/mL of primocin and 1 mg/mL of type I collagenase were added thereto, and allowed to react at 37° C. for 2 hours. After completion of the reaction, precipitated cells were obtained by centrifugation (2000 rpm, 5 minutes), and the cells were suspended in a culture medium (DMEM medium containing 0.2% primocin, 1% glutamax, and 10% FBS) and filtered, and then centrifuged (1000 rpm, 5 minutes) to obtain precipitated cells. The obtained cells were added to a lysing buffer (ACK lysing buffer, Gibco) and allowed to react for 1 minute. Subsequently, the cells (1.89×10⁵ cells) were washed with PBS, and then inoculated in a K-NAC medium (keratinocyte-SFM medium containing 5% FBS, 1% of 20 mM ascorbic acid, and 0.5% of 400 mM N-acetyl-L-cysteine), and then cultured under conditions of 37° C. and 5% CO₂ for 48 hours to obtain cultured cells. The obtained cells were inoculated in a serum-free medium (H1 medium, DMEM medium), and cultured for 96 hours to obtain a human adipose-derived stem cell culture medium.

### EXAMPLE 2

### Effect of Cell Culture Medium on Proliferation Ability of Fibroblast

Fibroblasts (HDFs) were seeded in a 96-well plate at a density of 1×10³ cells per well and cultured for 24 hours. Next, the fibroblast culture medium, the human umbilical cord blood-derived mesenchymal stem cell culture medium obtained in Example 1-1, or the human adipose-derived stem cell culture medium obtained in Example 1-2 were added thereto and cultured for 72 hours. In this regard, H1 medium was added to fibroblasts, which was used as a control group. After completion of the culture, 10 μL CCK-8 in a CCK-8 kit was added to the culture and allowed to react for 3 hours. Then, absorbance at 450 nm was measured to determine and compare the proliferation abilities of fibroblasts (FIG. 1).

FIG. 1 shows photographs (A) showing the results of comparing the effects of the control group (CTL), the fibroblast culture medium (HDF CM), the human adipose-derived stem cell culture medium (AD-MSC CM), or the human umbilical cord blood-derived mesenchymal stem cell culture medium (UCB-MSC CM) on proliferation ability of fibroblasts, a graph (B) showing absorbance, a graph (C) showing the number of cells, and a graph (D) showing a total protein expression level. As shown in FIG. 1, it was confirmed that the human umbilical cord blood-derived mesenchymal stem cell culture medium (UCB-MSC CM) may further promote fibroblast proliferation and may also increase a total amount of proteins secreted from the cells, as compared with the fibroblast culture medium (HDF CM) or the human adipose-derived stem cell culture medium (AD-MSC CM).

### EXAMPLE 3

### Analysis of Migration of Fibroblast

Fibroblasts were seeded in a 6-well plate at a density of 2×10⁵ cells per well, and cultured for 48 hours. Then, the medium was removed, and the bottom of the culture plate was scratched and washed with PBS. Subsequently, the fibroblast culture medium, the human umbilical cord blood-derived mesenchymal stem cell culture medium obtained in Example 1-1, or the human adipose-derived stem cell culture medium obtained in Example 1-2 were added thereto and cultured for 72 hours. In this regard, H1 medium was added to the fibroblasts, which was used as a control group. After completion of the culture, the level of fibroblasts that migrated into the scratch area was observed under a microscope (FIG. 2).

FIG. 2 shows microscopic images showing the results of comparing the effects of the control group (CTL), the fibroblast culture medium (HDF CM), the human adipose-derived stem cell culture medium (AD-MSC CM), or the human umbilical cord blood-derived mesenchymal stem cell culture medium (UCB-MSC CM) on migration of fibroblasts. As shown in FIG. 2, it was confirmed that the human umbilical cord blood-derived mesenchymal stem cell culture medium (UCB-MSC CM) may promote fibroblast migration, as compared with the fibroblast culture medium (HDF CM) or the human adipose-derived stem cell culture medium (AD-MSC CM).

### EXAMPLE 4

### Analysis of Matrix Protein Expression of Fibroblasts

### EXAMPLE 4-1

### Western Blot Analysis

Fibroblasts were seeded in a 6-well plate at a density of 2×10⁵ cells per well and cultured for 24 hours. Subsequently, the fibroblast culture medium, the human umbilical cord blood-derived mesenchymal stem cell culture medium obtained in Example 1-1, or the human adipose-derived stem cell culture medium obtained in Example 1-2 were added thereto, and cultured for 24 hours. Each of the cultured fibroblasts was subjected to Western blot analysis using antibodies against type I collagen (collagen I), type 4 collagen (collagen IV), fibronectin, or elastin (FIG. 3A). In this regard, GAPDH was used as an internal control group.

FIG. 3A shows a Western blot analysis image showing the results of comparing the effects of the control group (CTL), the fibroblast culture medium (HDF CM), the human adipose-derived stem cell culture medium (AD-MSC CM), or the human umbilical cord blood-derived mesenchymal stem cell culture medium (UCB-MSC CM) on protein expression levels of various matrix proteins expressed in the fibroblasts. As shown in FIG. 3A, it was confirmed that fibronectin and elastin were expressed at the highest levels in the fibroblasts treated with the human umbilical cord blood-derived mesenchymal stem cell culture medium (UCB-MSC CM).

### EXAMPLE 4-2

### RT-PCR Analysis

Total RNA was obtained from each of the fibroblasts cultured in Example 4-1, and cDNA was synthesized by using RT-Premix (Bioneer). The synthesized cDNA as a template and the following primers were used to perform PCR, and expression levels of type I collagen (Type I collagen), type III collagen (Type III collagen), and fibronectin were compared at an mRNA level (FIG. 4B). In this regard, GAPDH was used as an internal control group.

Collagen type I F:
(SEQ ID NO: 1)
5′-tcaaggtttccaaggacctg-3′
Collagen type I R:
(SEQ ID NO: 2)
5′-tcaaggtttccaaggacctg-3′
Collagen type III F:
(SEQ ID NO: 3)
5′-aaaggggagctggctacttc-3′
Collagen type III R:
(SEQ ID NO: 4)
5′-gcgagtaggagcagttggag-3′
Fibronectin F:
(SEQ ID NO: 5)
5′-tgaagaggggcacatgctga-3′
Fibronectin R:
(SEQ ID NO: 6)
5′-gtgggagttgggctgactcg-3′

FIG. 3B shows an RT-PCR analysis image showing the results of comparing the effects of the control group (CTL), the fibroblast culture medium (HDF CM), the human adipose-derived stem cell culture medium (AD-MSC CM), or the human umbilical cord blood-derived mesenchymal stem cell culture medium (UCB-MSC CM) on expression levels of various matrix proteins expressed in the fibroblasts. As shown in FIG. 3B, it was confirmed that type III collagen and fibronectin were expressed at the highest levels in the fibroblasts treated with the human umbilical cord blood-derived mesenchymal stem cell culture medium (UCB-MSC CM).

### EXAMPLE 5

### Analysis of Therapeutic Effects on Skin Wound Animal Model

First, a 6 mm full thickness skin wound was made on the back of 5-week-old nude mouse by using a biopsy punch to prepare a skin wound animal model.

24 hours later, 200 μL of each of the fibroblast culture medium, the human umbilical cord blood-derived mesenchymal stem cell culture medium obtained in Example 1-1, or the human adipose-derived stem cell culture medium obtained in Example 1-2 was applied to the wound area of the prepared skin wound animal model, and each culture medium was fixed on the wound area using a silicone band. 72 hours later, the same procedure was repeated. In this regard, the wound animal model to which H1 medium was applied was used as a control group. After application, the wound animal models were raised for 7 days, and then a reduction in the wound size was compared (FIG. 4A).

FIG. 4A shows a graph and an image showing the results of comparing the therapeutic effects of the control group (CTL), the fibroblast culture medium (HDF CM), the human adipose-derived stem cell culture medium (AD-MSC CM), or the human umbilical cord blood-derived mesenchymal stem cell culture medium (UCB-MSC CM). As shown in FIG. 4A, it was confirmed that the human umbilical cord blood-derived mesenchymal stem cell culture medium (UCB-MSC CM) showed the most excellent wound therapeutic effect.

Meanwhile, the wound areas of the animal models were excised, and the cross-sections of the wound areas were compared (FIG. 4B).

FIG. 4B shows tissue images showing the results of comparing the wound areas of wound animal models, each animal treated with the control group (CTL), the fibroblast culture medium (HDF CM), the human adipose-derived stem cell culture medium (AD-MSC CM), or the human umbilical cord blood-derived mesenchymal stem cell culture medium (UCB-MSC CM). As shown in FIG. 4B, it was also confirmed that the human umbilical cord blood-derived mesenchymal stem cell culture medium (UCB-MSC CM) showed the most excellent wound therapeutic effect.

### EXAMPLE 6

### Analysis of GDF11 (Growth Differentiation Factor 11)

### EXAMPLE 6-1

### RT-PCR Analysis of GDF11

Total RNAs were obtained from the fibroblasts cultured using H1 medium, the human umbilical cord blood-derived mesenchymal stem cells obtained in Example 1-1, or the human adipose-derived stem cells obtained in Example 1-2, and each cDNA was synthesized therefrom. The synthesized cDNA as a template and the following primers were used to perform real-time qPCR and PCR, and mRNA levels of GDF11 were compared (FIG. 5A and 5B). In this regard, RPL13A was used as an internal control group.

GDF11 F:
(SEQ ID NO: 7)
5′-gatcctggacctacacgacttc-3′
GDF11 R:
(SEQ ID NO: 8)
5′-ggccttcagtacctttgtgaac-3′
RPL13A F:
(SEQ ID NO: 9)
5′-gcacgaccttgagggcagcc-3′
RPL13A R:
(SEQ ID NO: 10)
5′-catcgtggctaaacaggtactg-3′

FIG. 5A shows a graph showing RT-PCR and real-time qPCR results of comparing GDF11 mRNA expression levels in fibroblasts (HDF), human adipose-derived stem cells (AD-MSC), or human umbilical cord blood-derived mesenchymal stem cells (UCB-MSC). FIG. 5B shows a photograph showing the result of RT-PCR. As shown in FIGS. 5A and 5B, it was confirmed that a large amount of GDF11 was expressed in the human umbilical cord blood-derived mesenchymal stem cells (UCB-MSC).

### EXAMPLE6-2

### Western Blot Analysis of GDF11

The fibroblast culture medium, the human umbilical cord blood-derived mesenchymal stem cell culture medium obtained in Example 1-1, and the human adipose-derived stem cell culture medium obtained in Example 1-2 were filtered (0.22 μm syringe filter), and then each of the culture media was concentrated. Each concentrated culture medium was subjected to Western blot analysis using an antibody against GDF11 (FIGS. 6A and 6B).

FIG. 6A shows Western blot analysis images showing the results of comparing levels of GDF11 in the human bone marrow-derived stem cell culture medium (BM-MSC CM) used as a control group (CTL), the human adipose-derived stem cell culture medium (AD-MSC CM), or the human umbilical cord blood-derived stem cell culture medium (UCB-MSC CM), and FIG. 6B shows a quantification graph of the Western blot analysis results. As shown in FIGS. 6A and 6B, it was confirmed that a relatively high level of GDF11 was included in the human umbilical cord blood-derived mesenchymal stem cell culture medium (UCB-MSC CM).

### EXAMPLE 7

### Functional Analysis of GDF11 in Human Umbilical Cord Blood-Derived Mesenchymal Stem Cells

### EXAMPLE 7-1

### Functional Analysis of GDF11 on Proliferation of Human Umbilical Cord Blood-Derived Mesenchymal Stem Cells

Human umbilical cord blood stem cells were seeded in a 6-well plate at a density of 2×10⁵ cells per well and cultured for 24 hours. Thereafter, the cultured cells were treated with 25 nM of each of control siRNA or GDF11 siRNA (siGDF11) and cultured for 72 hours. Subsequently, each of the cells was sub-cultured, and then respectively treated with siRNA under the same conditions, followed by culturing. After completion of the culturing, the cells were collected and seeded in a 24-well plate at a density of 4×10⁴ cells per well and cultured for 2 days. According to the method of Example 2, proliferation abilities of the cells were compared (FIG. 7A). According to the methods of Examples 4-1 and 6-1, mRNA levels of GDF11, type I collagen (collagen I), and type III collagen (collagen III) expressed in each of the cells were compared (FIG. 7B). In this regard, RPL13A was used as an internal control group.

FIG. 7A shows a graph showing the effect of GDF11 on proliferation of human umbilical cord blood-derived mesenchymal stem cells, and FIG. 7B shows a photograph showing the result of comparing changes in the collagen expression level according to suppression of GDF11 expression in the human umbilical cord blood-derived mesenchymal stem cells. As shown in FIG. 7A, it was confirmed that when GDF11 expression was decreased, the proliferation rate of the human umbilical cord blood-derived mesenchymal stem cells was decreased. As shown in FIG. 7B, it was confirmed that when GDF11 expression was decreased, the expression level of type III collagen in the human umbilical cord blood-derived mesenchymal stem cells was decreased.

### EXAMPLE 7-2

### Expression Mechanism Analysis of GDF11 in Human Umbilical Cord Blood-Derived Mesenchymal Stem Cells

Human umbilical cord blood stem cells were seeded in a 100 mm culture plate at a density of 5×10⁵ cells per well, and when confluency reached 80% to 90%, the cells were seeded in a 6-well plate at a density of 2×10⁵ cells per well and cultured for 24 hours.

Subsequently, the medium was replaced by a DMEM medium containing 100X glutamax, and treated with EGF, bFGF, vEGF, or TGF-betal at a concentration of 1 ng/mL or 10 ng/mL, followed by culturing for 1 day, 3 days, and 6 days. After completion of the culturing, total RNA was isolated from each of the cells, and cDNA was synthesized therefrom. Real-time PCR was performed by using a SYBR Green PCR amplifier (FIGS. 8A to 8E). In this regard, cells treated with H1 medium were used as a control group.

FIG. 8 shows graphs showing the result of comparing changes in the GDF11 expression level in the human umbilical cord blood-derived mesenchymal stem cells treated with the control group (a), EGF (b), bFGF (c), TGF-betal (d), or vEGF (e) according to treatment time and concentration. As shown in FIG. 8, in all of the cases, GDF11 expression was increased, and the highest expression was observed at 6 days. It was also confirmed that when the above four factors were treated at the same time, the expression was further increased.

These results suggest that the above four factors are involved in the regulation of GDF11 expression in umbilical cord blood-derived mesenchymal stem cells.

### EXAMPLE 8

### Functional Analysis of GDF11 in Fibroblasts

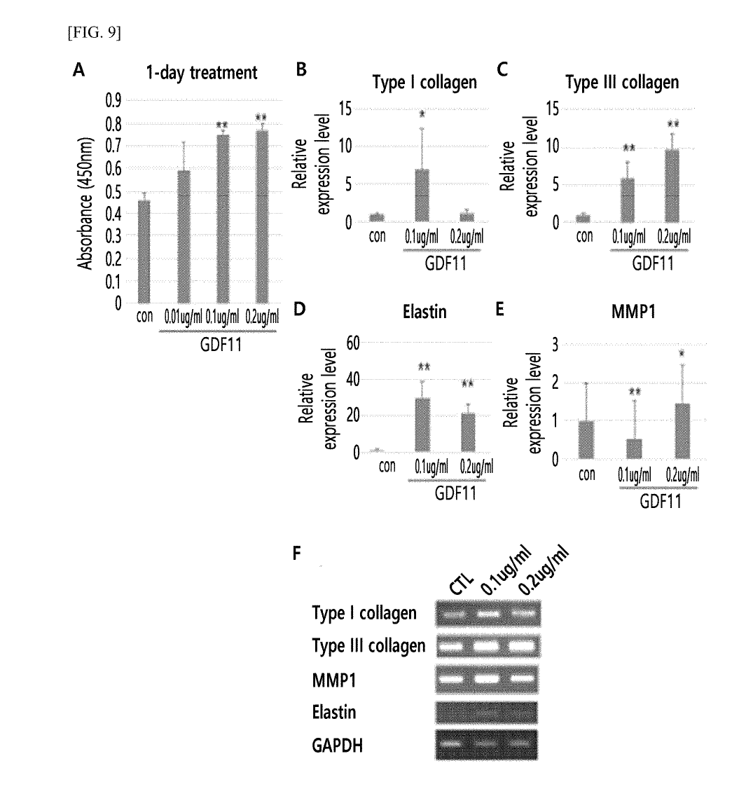

During culturing, fibroblasts were treated with 0 μg/mL, 0.01 μg/mL, 0.1 μg/mL, or 0.2 μg/mL of GDF11, and after completion of the culturing, a proliferation level of the fibroblasts, an expression level of type I collagen expressed in the fibroblasts, an expression level of type III collagen expressed in the fibroblasts, an expression level of elastin expressed in the fibroblasts, and an expression level of MMP1 expressed in the fibroblasts were compared and analyzed (FIG. 9).

FIG. 9 shows graphs and a photograph showing the results of comparing the fibroblast proliferation levels in the GDF11-treated fibroblasts (FIG. 9A), the expression levels of type I collagen expressed in the fibroblasts (FIGS. 9B and 9F), the expression levels of type III collagen expressed in the fibroblasts (FIGS. 9C and 9F), the expression levels of elastin expressed in the fibroblasts (FIGS. 9D and 9F), and the expression levels of MMP1 expressed in the fibroblasts (FIGS. 9E and 9F). As shown in FIG. 9A, it was confirmed that as the GDF11 concentration was increased, the fibroblast proliferation was promoted. As shown in FIGS. 9B and 9F, it was confirmed that as the GDF11 concentration was increased, the type I collagen expression was promoted, but the excessive increase of the concentration actually inhibited the expression. As shown in FIGS. 9C and 9F, it was confirmed that as the GDF11 concentration was increased, the type III collagen expression was promoted. As shown in FIGS. 9D and 9F, it was confirmed that as the GDF11 concentration was increased, the elastin expression was promoted, but the excessive increase of the concentration actually inhibited the expression. As shown in FIGS. 9E and 9F, it was confirmed that as the GDF11 concentration was increased, the MMP1 expression was inhibited, but the excessive increase of the concentration actually increased the expression.

These results suggest that GDF11 may exhibit the effect of promoting skin regeneration and wrinkle improvement in which collagen and elastin are involved.

1. A method of treating wounds comprising the step of administering to a subject a composition comprising GDF11 (growth differentiation factor 11) or a human-derived adult stem cell culture medium including the same.

2. The method of claim 1, wherein the human-derived adult stem cells are derived from umbilical cord, umbilical cord blood, bone marrow, fat, muscle, nerve, skin, amniotic membrane, or placenta.

3. The method of claim 1, wherein the composition further comprises a pharmaceutically acceptable carrier, excipient, or diluent.

4. The method of claim 1, wherein the composition promotes fibroblast proliferation.

5. A method of regenerating skin comprising the step of administering to a subject a composition including GDF11 (growth differentiation factor 11) or a human-derived adult stem cell culture medium including the same.

6. The method of claim 5, wherein the human-derived adult stem cells are derived from umbilical cord, umbilical cord blood, bone marrow, fat, muscle, nerve, skin, amniotic membrane, or placenta.

7. The method of claim 5, wherein the composition further comprises a pharmaceutically acceptable carrier, excipient, or diluent.

8. The method of claim 5, wherein the composition promotes fibroblast proliferation.

9. A method of improving wrinkles comprising the step of administering to a subject a composition including GDF11 (growth differentiation factor 11) or a human-derived adult stem cell culture medium including the same.

10. The method of claim 9, wherein the human-derived adult stem cells are derived from umbilical cord, umbilical cord blood, bone marrow, fat, muscle, nerve, skin, amniotic membrane, or placenta.

11. The method of claim 9, wherein the composition further comprises a pharmaceutically acceptable carrier, excipient, or diluent.

12. The method of claim 9, wherein the composition promotes fibroblast proliferation.

13. A method of culturing fibroblasts comprising the step of inoculating and culturing fibroblasts in the medium composition for culturing fibroblasts, wherein the composition comprises GDF11 (growth differentiation factor 11) or a human-derived adult stem cell culture medium including the same.

14. The method of claim 13, wherein the human-derived adult stem cells are derived from umbilical cord, umbilical cord blood, bone marrow, fat, muscle, nerve, skin, amniotic membrane, or placenta.

15. A method of preparing GDF11 comprising the steps of:
(a) culturing human-derived adult stem cells to obtain a culture supernatant; and
(b) collecting GDF11 (growth differentiation factor 11) from the obtained culture supernatant.

16. The method of claim 15, wherein the human-derived adult stem cells are derived from umbilical cord, umbilical cord blood, bone marrow, fat, muscle, nerve, skin, amniotic membrane, or placenta.

17-19. (canceled)
